(19) United States
(12) Patent Application Publication
(10) Pub. No.: US 2024/0366562 A1
(43) Pub. Date: Nov. 7, 2024

(54) GYNECOMASTIA AND/OR MASTALGIA TREATMENT

(71) Applicant: Ilan Chaitowitz, Greater London (GB)

(72) Inventor: Ilan Chaitowitz, Greater London (GB)

(21) Appl. No.: 18/574,806

(22) PCT Filed: Jun. 28, 2022
(86) PCT No.: PCT/EP2022/067796

§ 371 Date: Dec. 28, 2023

(30) Foreign Application Priority Data
Jun. 29, 2021 (GB) 2109385.1
May 8, 2022 (GB) 2206703.7

(51) Int. Cl. A61K 31/404; A61K 31/138; A61K 31/352; A61K 31/585; A61K 36/185; A61K 36/31; A61K 36/48; A61K 36/87; A61K 36/9066; A61P 5/24

(52) U.S. Cl. CPC A61K 31/404; A61K 31/138; A61K 31/352; A61K 31/585; A61K 36/185; A61K 36/31; A61K 36/48; A61K 36/87; A61K 36/9066; A61P 5/24

(57) ABSTRACT

A selective estrogen receptor modulator and/or an aromatase inhibitor (A) for use in a method of preventing or treating gynecomastia and/or mastalgia,
wherein said method comprises administering said selective estrogen receptor modulator and/or aromatase inhibitor in combination with
(B) an estrogen modulator, wherein said estrogen modulator is different to said selective estrogen receptor modulator.

### SUMMARY OF THE INVENTION

The present invention relates to combination therapies for use in the treatment of gynecomastia and/or mastalgia. The present invention relates more particularly to the combination of (A) a selective estrogen receptor modulator and/or an aromatase inhibitor, with (B) an estrogen modulator, for use in the treatment of gynecomastia and/or mastalgia. The invention also relates to pharmaceutical compositions comprising said compounds, to methods of treating or preventing gynecomastia and/or mastalgia, and to the use of the compounds in the manufacture of a medicament for the treatment or prevention of gynecomastia and/or mastalgia.

### BACKGROUND TO THE INVENTION

Gynecomastia is a breast disorder that affects about 60% of men worldwide. Gynecomastia can be due to weight gain, drug use, anabolic steroids, or other underlying medical conditions. It may result in “female-like breasts”. The male breast enlargement can affect psychological, social, and emotional well-being.

The stigma is why many men seek professional help. Luckily, today, gynecomastia can be treated by male breast reduction surgery. Furthermore, several types of surgery are available to treat gynecomastia. The surgical technique depends on the gynecomastia grade or the severity of the breast enlargement. The various surgical procedures can produce successful and effective results.

But, not all men are ready to undergo surgery, and not all men are good candidates or even want a surgical solution. Moreover, poor surgical outcomes are commonplace as up to one third of patients are unsatisfied with theirs. Gynecomastia surgery risks include: anesthesia risks; bleeding (hematoma), blood clots, breast asymmetry, breast contour and shape irregularities, changes in nipple or breast sensation may be temporary or permanent, damage to deeper structures—such such as nerves, blood vessels, muscles and lungs—can occur and may be temporary or permanent, deep vein thrombosis, cardiac and pulmonary complications, fatty tissue found in the breast might die (fat necrosis), fluid accumulation (seroma), infection-inverted nipples, persistent pain, poor wound healing, possibility of revision surgery, reactions to tape, suture materials, glues, topical preparations or injected agents, unacceptable scarring.

Most men would prefer a medication that solved the breast gland problem. Because of this, studies have been done to find a medical solution. Some of these studies revolve around selective estrogen receptor modulator (SERM) Tamoxifen. Many professionals consider the option of Tamoxifen for the treatment of gynecomastia to be promising.

The enlarged breasts in patients suffering from gynecomastia is typically due to breast tissue growth caused as a result of a hormonal imbalance between estrogen and androgen. The balance between the hormones is often caused by an increase in estrogen production or abnormally high levels of estrogen. It is therefore desirable to target estrogen production in gynecomastia treatment.

Mastalgia, also known as mastodynia, is a common breast disorder that affects up to 70% of women at some point in their life and especially among women aged 30 to 50. It is a common reason for women's visits to their doctors. In men, it is commonly caused by gynecomastia, which, as mentioned above, is a breast disorder that affects about 60% of men worldwide. Mastalgia can be due to menstruation, weight gain, drug use, anabolic steroids, or other underlying medical conditions. Breast pain is a significant issue within the general population as it can affect quality of life, daily activities, sleep, psychological, social, and emotional well-being (Scurr et al. 2014).

For women with mastalgia, topical non-steroidal anti-inflammatory drugs (NSAIDs) are effective in relieving breast pain and should be considered as a first-line treatment. However, these treatments are not always effective.

In transgender women, hormone therapy may cause breast pain. In transgender men, breast pain may be caused by the minimal amount of breast tissue that may remain after a mastectomy.

Danazol is the only prescription medication approved by the United States Food and Drug Administration for treating fibrocystic breasts. However, danazol carries the risk of potentially severe side effects, such as heart and liver problems, as well as weight gain and voice changes. Tamoxifen, a prescription medication for breast cancer treatment and prevention, may help, but this drug also carries the potential for side effects that may be more bothersome than the breast pain itself.

For men with gynecomastia, the stigma is why many men seek professional help. Luckily, today, gynecomastia-related mastalgia can be treated by male breast reduction surgery. Furthermore, several types of surgery are available to treat gynecomastia. The surgical technique depends on the mastalgia grade or the severity of the breast enlargement. The various surgical procedures can produce successful and effective results. As mentioned above, however, surgery is often not desirable/appropriate.

Most women and men would prefer a medication that solved the breast gland problem quickly and with few, if any, side effects. Because of this, studies have been done to find a medical solution. Some of these studies revolve around selective estrogen receptor modulator (SERM) Tamoxifen. Many professionals consider the option of Tamoxifen for the treatment of mastalgia to be promising.

Mastalgia is typically due to breast tissue growth caused as a result of a hormonal imbalance between estrogen and androgen. As in gynecomastia, the imbalance between the hormones is often caused by an increase in estrogen production or abnormally high levels of estrogen. The two conditions often occur together. It is therefore desirable to target estrogen production in mastalgia treatment.

Tamoxifen is the oldest and most-prescribed selective estrogen receptor modulator (SERM). Tamoxifen is currently approved by the U. S. Food and Drug Administration (FDA) to treat:
1) women and men diagnosed with hormone-receptor-positive, early-stage breast cancer after surgery (or possibly chemotherapy and radiation) to reduce the risk of the cancer coming back (recurring).
2. Women and men diagnosed with advanced-stage or metastatic hormone-receptor-positive disease.
3. Reduce breast cancer risk in women who haven't been diagnosed but are at higher-than-average risk for the disease.

Tamoxifen works by interfering with the effects of estrogen in the breast tissue. Tamoxifen is the most prescribed selective estrogen receptor modulator (SERM). Tamoxifen can either block or activate the function of the female hormone estrogen. In the case of the breast cells, the estrogen will block this action.

In breast cancer prevention, Tamoxifen is known to have a dual mechanism of action: (1) to compete with 17β-estradiol (E2) at the receptor site and to block the promotional role of E2 in breast cancer; and (2) to bind DNA after metabolic activation and to initiate carcinogenesis.

Besides the oncology setting, Tamoxifen is used in endocrine therapy. This treatment can influence the effects of estrogen in the cancer cells of the breast. This action, in turn, reduces the risk of the development of this cancer.

Furthermore, people with dense breasts use Tamoxifen to reduce their breasts' density.

Tamoxifen can help prevent the development of breast tissue, hence why it is seen as a promising candidate for gynecomastia and/or mastalgia treatment (but has not been approved by regulatory bodies for gynecomastia or mastalgia treatment).

The present inventor sought alternative and/or improved methods for preventing or treating gynecomastia and/or mastalgia. The present inventor has surprisingly found that the combination of (A) a SERM (e.g. Tamoxifen) and/or AI with (B) an estrogen modulator (different to the SERM and different to the AI) such as indole-3-carbinol (I3C), offers promising results in gynecomastia and/or mastalgia treatment. The two components can work via different biochemical pathways and therefore offer cooperative actions on estrogen production and a combinatorial treatment of gynecomastia and/or mastalgia.

Moreover, certain SERMs or AIs can cause significant side effects. For example, Tamoxifen side effects include the following: headaches, nausea, hot flashes, skin rash, fatigue, sexual dysfunction, and weight and mood changes. The use of a second estrogen modulator (B) offers the possibility of reducing the dose of the SERM (e.g. Tamoxifen) and/or AI during gynecomastia and/or mastalgia treatment, resulting in a reduction of side effects.

Moreover, reducing the dose of the SERM/AI offers the possibility of reducing the likelihood of SERM/AI resistance.

It has been shown that combinations of I3C and the anti-estrogen tamoxifen cooperate to inhibit the growth of the estrogen-dependent human MCF-7 breast cancer cell line more effectively than either agent alone (Cover et al., ‘Indole-3-Carbinol and Tamoxifen Cooperate to Arrest the Cell Cycle of MCF-7 Human Breast Cancer Cells’. J Cancer Research. 1244-1251, 59 (1999)). Several lines of evidence suggest that I3C works through a mechanism distinct from tamoxifen. For example, I3C has been shown to fail to compete with estrogen for estrogen receptor binding, and it specifically down-regulates the expression of CDK6.

These results demonstrate that I3C and Tamoxifen work through different signal transduction pathways to suppress the growth of human breast cancer cells.

Given the idiosyncratic features of the growth suppression cascades induced by I3C and tamoxifen, Cover et al. (1999) demonstrated that a combination of tamoxifen and I3C displayed a more effective growth suppression response, a more stringent inhibition of CDK2 specific activity, and more endogenous Rb phosphorylation than either compound alone.

Similarly, Malejka-Giganti et al. (2007) found that in rats treated with both Tamoxifen and I3C, suppression of mammary carcinogenesis by I3C did not weaken but fostered the benefits of chemoprevention with Tamoxifen. This was evident from the significant decreases in the mean tumour number per rat that indicated a cooperative effect of the two compounds

However, in the only randomized, placebo-controlled trial of diindolylmethane, the main active metabolite from the breakdown of indole-3-carbinol, for breast cancer biomarker modulation in women taking Tamoxifen, Thomson et al. (2017) found that the combination was associated with decreases in Tamoxifen metabolites. It therefore appears that DIM attenuates the clinical benefit of Tamoxifen. Thomson et al. urges caution against using DIM in combination with Tamoxifen since DIM might reduce the effectiveness of Tamoxifen.

A surprising aspect of the present invention is the realisation that SERMs and/or AIs in combination with different estrogen modulators represent a combinatorial therapy for gynecomastia and/or mastalgia.

### SUMMARY OF THE INVENTION

In a first aspect, the invention provides a selective estrogen receptor modulator and/or an aromatase inhibitor (A) for use in a method of preventing or treating gynecomastia and/or mastalgia,
wherein said method comprises administering said selective estrogen receptor modulator and/or aromatase inhibitor in combination with
(B) an estrogen modulator, wherein said estrogen modulator is different to said selective estrogen receptor modulator.

In a further aspect, the invention provides a selective estrogen receptor modulator and/or an aromatase inhibitor (A) for use in a method of preventing or treating gynecomastia and/or mastalgia,
wherein said method comprises administering said selective estrogen receptor modulator and/or aromatase inhibitor in combination with
(B) a cruciferous vegetable, wild nettle root, chrysin, soy, turmeric, maca, flavonoids or grape seed supplement or extract, preferably a cruciferous vegetable supplement or extract.

In a further aspect, the invention provides a composition comprising
(A) a selective estrogen receptor modulator and/or an aromatase inhibitor;
(B) an estrogen modulator, wherein said estrogen modulator is different to said selective estrogen receptor modulator;
for use in the treatment or prevention of gynecomastia and/or mastalgia.

In a further aspect, the invention provides a pharmaceutical composition for combination therapy for treating gynecomastia and/or mastalgia, comprising
(A) a first pharmaceutical component comprising a selective estrogen receptor modulator and/or an aromatase inhibitor;
(B) a second pharmaceutical component comprising an estrogen modulator, wherein said estrogen modulator is different to said selective estrogen receptor modulator as active ingredients.

In a further aspect, the invention provides Tamoxifen, or a pharmaceutically acceptable salt, solvate or hydrate thereof, for use in a method of preventing or treating gynecomastia and/or mastalgia,
wherein said method comprises administering said Tamoxifen in combination with
(B) Indole-3-carbinole (I3C) and/or 3,3′-diindolylmethane (DIM), or pharmaceutically acceptable salts, solvates or hydrates thereof.

In a further aspect, the invention provides a combination of
(A) a selective estrogen receptor modulator and/or an aromatase inhibitor, preferably wherein said selective estrogen receptor modulator and/or aromatase inhibitor is a selective estrogen receptor modulator selected from Tamoxifen, Raloxifene, and Clomifene, or pharmaceutically acceptable salts, solvates or hydrates thereof
(B) an estrogen modulator different to said selective estrogen receptor modulator, preferably wherein said estrogen modulator is selected from Indole-3-carbinole (I3C), 3,3′-diindolylmethane (DIM), 5,11-dihydroindolo-[3,2-b]carbazole (ICZ), and/or 5,6,11,12,17,18-hexahydrocyclononal [1,2-b:4,5-b′:7,8-b″] triindole (CT), or pharmaceutically acceptable salts, solvates or hydrates thereof.
for simultaneous, separate, or sequential use in the treatment of gynecomastia and/or mastalgia.

In a further aspect, the invention provides a medicament comprising
(A) a selective estrogen receptor modulator and/or an aromatase inhibitor, preferably wherein said selective estrogen receptor modulator and/or aromatase inhibitor is a selective estrogen receptor modulator selected from Tamoxifen, Raloxifene, and Clomifene, or pharmaceutically acceptable salts, solvates or hydrates thereof
(B) an estrogen modulator different to said selective estrogen receptor modulator, preferably wherein said estrogen modulator is selected from Indole-3-carbinole (I3C), 3,3′-diindolylmethane (DIM), 5,11-dihydroindolo-[3,2-b]carbazole (ICZ), and/or 5,6,11,12,17,18-hexahydrocyclononal [1,2-b:4,5-b′:7,8-b″] triindole (CT), or pharmaceutically acceptable salts, solvates or hydrates thereof,
as a pharmaceutical combination for simultaneous, separate or sequential use in the treatment of gynecomastia and/or mastalgia.

In a further aspect, the invention provides a kit comprising
(A) a selective estrogen receptor modulator and/or an aromatase inhibitor, preferably wherein said selective estrogen receptor modulator and/or aromatase inhibitor is a selective estrogen receptor modulator selected from Tamoxifen, Raloxifene, and Clomifene, or pharmaceutically acceptable salts, solvates or hydrates thereof; and
(B) an estrogen modulator different to said selective estrogen receptor modulator, preferably wherein said estrogen modulator is selected from Indole-3-carbinole (I3C), 3,3′-diindolylmethane (DIM), 5,11-dihydroindolo-[3,2-b]carbazol (ICZ), and/or 5,6,11,12,17,18-hexahydrocyclononal [1,2-b:4,5-b′:7,8-b″] triindole (CT), or pharmaceutically acceptable salts, solvates or hydrates thereof
for simultaneous, separate or sequential use in the treatment of gynecomastia and/or mastalgia.

In a further aspect, the invention provides a method of treating or preventing gynecomastia and/or mastalgia, comprising administering simultaneously, separately or sequentially in a subject in need thereof an effective amount of:
(A) a selective estrogen receptor modulator and/or an aromatase inhibitor, preferably wherein said selective estrogen receptor modulator and/or aromatase inhibitor is a selective estrogen receptor modulator selected from Tamoxifen, Raloxifene, and Clomifene, or pharmaceutically acceptable salts, solvates or hydrates thereof; and
(B) an estrogen modulator different to said selective estrogen receptor modulator, preferably wherein said estrogen modulator is selected from Indole-3-carbinol (I3C), 3,3′-diindolylmethane (DIM), 5,11-dihydroindolo-[3,2-b]carbazole (ICZ), and/or 5,6,11,12,17,18-hexahydrocyclononal [1,2-b:4,5-b′:7,8-b″] triindole (CT), or pharmaceutically acceptable salts, solvates or hydrates thereof.

In a further aspect, the invention provides the use of
(A) a selective estrogen receptor modulator and/or an aromatase inhibitor, preferably wherein said selective estrogen receptor modulator and/or aromatase inhibitor is a selective estrogen receptor modulator selected from Tamoxifen, Raloxifene, and Clomifene, or pharmaceutically acceptable salts, solvates or hydrates thereof; and
(B) an estrogen modulator different to said selective estrogen receptor modulator, preferably wherein said estrogen modulator is selected from Indole-3-carbinole (I3C), 3,3′-diindolylmethane (DIM), 5,11-dihydroindolo-[3,2-b]carbazole (ICZ), and/or 5,6,11,12,17,18-hexahydrocyclononal [1,2-b:4,5-b′:7,8-b″] triindol (CT), or pharmaceutically acceptable salts, solvates or hydrates thereof in the manufacture of a combination medicament for the treatment of gynecomastia and/or mastalgia.

In a further aspect, the invention provides a pharmaceutical composition comprising:
(A) a selective estrogen receptor modulator and/or an aromatase inhibitor, preferably wherein said selective estrogen receptor modulator and/or aromatase inhibitor is a selective estrogen receptor modulator selected from Tamoxifen, Raloxifene, and Clomifene, or pharmaceutically acceptable salts, solvates or hydrates thereof; and
(B) an estrogen modulator different to said selective estrogen receptor modulator, preferably wherein said estrogen modulator is selected from Indole-3-carbinole (I3C), 3,3′-diindolylmethane (DIM), 5,11-dihydroindolo-[3,2-b]carbazole (ICZ), and/or 5,6,11,12,17,18-hexahydrocyclononal [1,2-b:4,5-b′:7,8-b″] triindole (CT), or pharmaceutically acceptable salts, solvates or hydrates thereof.

In a further aspect, the invention provides the use of:
A) a selective estrogen receptor modulator and/or an aromatase inhibitor, preferably wherein said selective estrogen receptor modulator and/or aromatase inhibitor is a selective estrogen receptor modulator selected from Tamoxifen, Raloxifene, and Clomifene, or pharmaceutically acceptable salts, solvates or hydrates thereof; and
(B) an estrogen modulator different to said selective estrogen receptor modulator, preferably wherein said estrogen modulator is selected from Indole-3-carbinole (I3C), 3,3′-diindolylmethane (DIM), 5,11-dihydroindolo-[3,2-b]carbazole (ICZ), and/or 5,6,11,12,17,18-hexahydrocyclononal [1,2-b:4,5-b′:7,8-b″] triindole (CT), or pharmaceutically acceptable salts, solvates or hydrates thereof
for reducing the breast size and/or breast pain of a subject.

In a further aspect the invention provides a method of reducing the breast size and/or breast pain of a subject, comprising a step of administering:
(A) a selective estrogen receptor modulator and/or an aromatase inhibitor, preferably wherein said selective estrogen receptor modulator and/or aromatase inhibitor is a selective estrogen receptor modulator selected from Tamoxifen, Raloxifene, and Clomifene, or pharmaceutically acceptable salts, solvates or hydrates thereof; and
(B) an estrogen modulator different to said selective estrogen receptor modulator, preferably wherein said estrogen modulator is selected from Indole-3-carbinole (I3C), 3,3′-diindolylmethane (DIM), 5,11-dihydroindolo-[3,2-b]carbazole (ICZ), and/or 5,6,11,12,17,18-hexahydrocyclononal [1,2-b:4,5-b′:7,8-b″]triindole (CT), or pharmaceutically acceptable salts, solvates or hydrates thereof;
wherein (A) and (B) are administered simultaneously, separately, or sequentially.

The features of the aspects and/or embodiments indicated herein are usable individually and in combination in all aspects and embodiments of the invention where technically viable, unless otherwise indicated.

### Ingredient (A)

‘SERM’ herein stands for selective estrogen receptor modulator. ‘AI’ herein stands for aromatase inhibitor. SERMs and AIs are well-established classes of compounds.

The SERM is typically different to the estrogen modulator (B). The SERM is typically different to the AI, and the AI is typically different to the estrogen modulator (B).

Any discussion relating to component/ingredient/compound/SERM/AI ‘A’ are applicable to all aspects and embodiments of the invention, where technically viable.

### Selective Estrogen Receptor Modulator (SERM)

Selective estrogen receptor modulators (SERMs) are a class of drugs that act on the estrogen receptor (ER). As their name indicates, their action is different in various tissues, and thus these substances are different from pure ER agonists and antagonists. As a result of this, they offer the possibility to selectively inhibit or stimulate estrogen-like action in various tissues.

In a particular embodiment, the SERM is a non-steroidal SERM. In a further particular embodiment, the SERM is selected from Tamoxifen, Clomifene and Raloxifene, or pharmaceutically acceptable salts, solvates or hydrates thereof. The compounds discussed below (whether Tamoxifen, Clomifene and Raloxifene, or other compounds) can be administered as pharmaceutically acceptable salts, solvates or hydrates thereof. In a particular embodiment, said SERM is a triphenylethylene-type SERM, i.e. is based on a triphenylethylene core. Alternatively put, the SERM can contain a triphenylethylene unit.

Tamoxifen has the following formula:

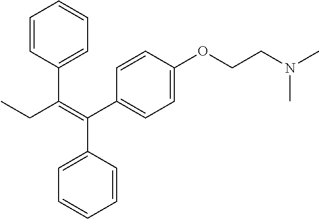 [SMILES: CC/C(=C(\c1ccccc1)c1ccc(OCCN(C)C)cc1)c1ccccc1]

Clomifene has the following formula:

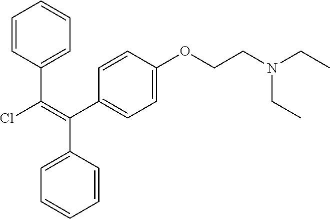 [SMILES: CCN(CC)CCOc1ccc(/C(=C(/Cl)c2ccccc2)c2ccccc2)cc1]

Raloxifene has the following formula:

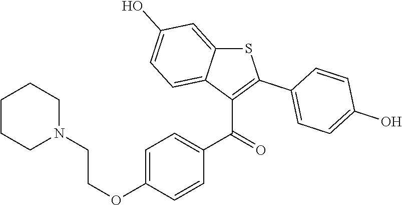 [SMILES: O=C(c1ccc(OCCN2CCCCC2)cc1)c1c(-c2ccc(O)cc2)sc2cc(O)ccc12]

In a particular embodiment, the SERM is a compound of the following formula:

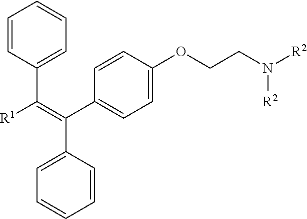

wherein R¹ is selected from H, halogen (preferably Cl), or C₁-C₆ alkyl (preferably C₁-C₂ alkyl, i.e. methyl or ethyl), preferably R¹ is selected from CI and ethyl; and each R² is independently selected from H, or C₁-C₆ alkyl (preferably C₁-C₂ alkyl, i.e. methyl or ethyl), preferably both R² groups are the same and are methyl or ethyl;
or pharmaceutically acceptable salts, solvates or hydrates thereof.

The structures of the SERMs tamoxifen and clomifene are similar and both have been suggested for the use in gynecomastia/mastalgia. Their biochemical/physiological properties are similar.

In all aspects and embodiments of the invention, it is preferred if the selective estrogen receptor modulator and/or aromatase inhibitor (A) is Tamoxifen, or a pharmaceutically acceptable salt, solvate or hydrate thereof.

### Tamoxifen

Tamoxifen is an FDA-approved prescription drug used to treat hormone-receptor-positive breast cancer. It has also been used to reduce the development of cancer in high-risk patients. Since its approval in 1998, Tamoxifen had already helped both women and men diagnosed with cancer of the breast. It has also shown promising results in the treatment of gynecomastia and mastalgia.

In the oncology setting, Tamoxifen works by preventing the development of breast cancer. Treatment and prevention are possible by interfering with the effects of estrogen in the glandular tissue.

For this reason, Tamoxifen is the most prescribed selective estrogen receptor modulator (SERM). It can either block or activate the function of estrogen on specific breast cells. Tamoxifen blocks the estrogen action on breast cells. Furthermore, Tamoxifen attaches to proteins or the hormone receptors in the cancer cells. The blockage of the hormone receptors, in effect, stops cancer from advancing.

Tamoxifen is used in conjunction with other medications that are undergoing hormonal treatment. Tamoxifen treatment can slow the progress of cancer cells by altering the body's hormone balance.

The rapid development of cancer is due to high estrogen levels. Tamoxifen lowers these estrogen levels and can slow the development of cancer and reduce tumor growth.

Because of how Tamoxifen works on stopping the development of cancerous cells, it has shown its efficacy. It can also reduce recurring and invasive breast carcinoma. Also, it can help lessen the chances of high-risk people developing the disease.

Gynecomastia is the development of breast tissue on a man's chest. This tissue results in the breast gland component of gynecomastia mixing with breast fat. While there are several reasons why men develop gynecomastia, it is a result of hormonal imbalance. This imbalance occurs when the estrogen levels become too high and the male hormone, testosterone, is lesser. In men mastalgia, or breast pain, often occurs concomitantly with gynecomastia as the two conditions can arise from the same underlying condition. In women, cyclical mastalgia is typically also caused by menstrual hormonal fluctuations.

Tamoxifen has both antiestrogenic and anti-tumor effects on glandular tissue. Researchers can use the “Tamoxifen effect” to determine if treatment is possible for gynecomastia and/or mastalgia. Tamoxifen may be beneficial in some cases, especially pubertal gynecomastia or mastalgia.

Whilst Tamoxifen provides a successful treatment and prevention for breast cancer, more research still needs to be done on antiestrogen compounds in other settings, e.g Tamoxifen in gynecomastia.

Tamoxifen is typically administered as a once or twice daily tablet. However, gel formulations of tamoxifen have been shown to significantly reduce side effects related to the oral formulation. A cationic nanoemulsion of Tamoxifen containing a charge inducer has been developed for improving biopharmaceutical attributes and anticancer potential of the drug. In a particular embodiment, the SERM and/or AI is administered as a topical formulation (e.g. cream, skin patch, gel etc.), or as a cationic nanoemulsion.

Raloxifene is a sister drug to Tamoxifen that would be a possible alternative to use with this formulation, along with other SERMs such as clomifene.

In breast tissue, tamoxifen typically acts as an ER antagonist so that transcription of estrogen-responsive genes is inhibited. The most abundant metabolites of tamoxifen in terms of circulating concentrations are N-desmethyltamoxifen, N,N-didesmethyltamoxifen, (Z)-endoxifen, and tamoxifen N-oxide.

### Aromatase Inhibitor

Aromatase inhibitors are another well-known class of compounds. Aromatase, also known as estrogen synthetase, is the key enzyme in estrogen biosynthesis. Aromatase inhibitors such as anastrozole and letrozole effectively delay epiphysial maturation in boys and improve testosterone levels in adult men. Therefore, aromatase inhibitors may be used to increase adult height in boys with gonadotropin-independent precocious puberty, idiopathic short stature, constitutional delay of puberty. may be used to increase gonadotropin secretion and thereby stimulate Leydig and Sertoli cell function. Aromatase inhibitors may be used to prevent or delay epiphysial closure and thereby increase adult height. Aromatase inhibitors may also be used to treat or prevent gynecomastia and/or mastalgia. A concern of aromatase inhibition is the possible detrimental effect on bone mineralization. Testolactone is considered to be a promising gynecomastia and/or mastalgia drug. Long-term efficacy and safety of the use of aromatase inhibitors has not yet been established in males, but their potential in anti-estrogen targeting is significant. It is therefore highly desirable to pursue combinatorial treatments, in which the dosage of any aromatase inhibitor could potentially be lowered, for example, or in which their anti-estrogen properties can be complimented.

In a particular embodiment, the aromatase inhibitor is selected from testolactone, anastrozole and letrozole, preferably testolactone, or pharmaceutically acceptable salts, solvates or hydrates thereof.

### Estrogen Modulator—Component (B)

The invention concerns the combination of
(A) a SERM and/or an AI with
(B) an estrogen modulator,
in the treatment of gynecomastia and/or mastalgia. The estrogen modulator (B) is typically a compound with an antiestrogen effect. It is different to said selective estrogen receptor modulator (SERM) and different to said aromatase inhibitor (AI). In the event that estrogen modulator (B) might be considered to be a SERM and/or an AI, the estrogen modulator (B) is different to the SERM and/or AI (A).

In a particular embodiment, the estrogen modulator is an estrogen inhibitor. In a particular embodiment, the estrogen modulator is an estrogen receptor antagonist. In a particular embodiment, the estrogen modulator (B) is an aromatase inhibitor (AI), preferably different to any aromatase inhibitor (A) In a particular embodiment, the estrogen modulator (B) is an inhibitor of enzyme CYP19A1 (a member of the cytochrome P450 family) (Eur J Nutr (2013) 52:1483-1492). In a particular embodiment, the estrogen modulator (B) is a downregulator of the expression of CYP19 in estrogen-responsive (ER+) breast cells, and/or is an agent that increases expression of CYP19 in estrogen-dependent (ER−) breast cells. In a particular embodiment, the estrogen modulator is able to bind aryl hydrocarbon receptor (AhR). In a particular embodiment, the estrogen modulator (B) affects estrogen production via a different biochemical pathway to the SERM and/or AI (A).

In a particular embodiment, the estrogen modulator in (B) is selected from the following compounds (i)-(iv):

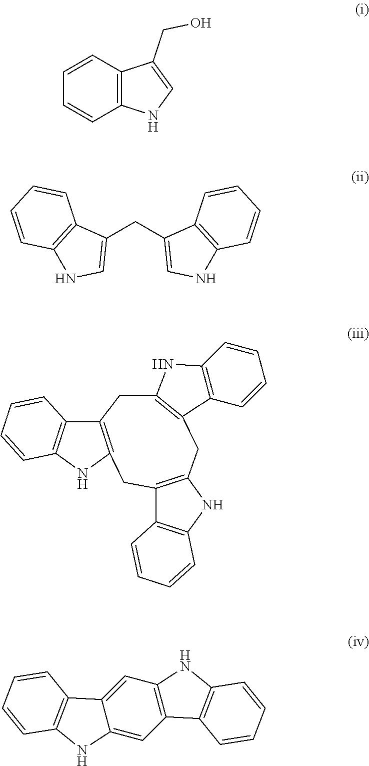 [SMILES: OCc1c[nH]c2ccccc12 ; c1ccc2c(Cc3c[nH]c4ccccc34)c[nH]c2c1 ; c1ccc2c3c([nH]c2c1)Cc1c([nH]c2ccccc12)Cc1c([nH]c2ccccc12)C3 ; c1ccc2c(c1)[nH]c1cc3c(cc12)[nH]c1ccccc13]

or pharmaceutically acceptable salts or hydrates thereof.

Compound (i) is Indole-3-carbinole (I3C); Compound (ii) is 3,3′-diindolylmethane (DIM); Compound (iii) is 5,6,11,12,17,18-hexahydrocyclononal [1,2-b:4,5-b′:7,8-b″]triindole (CT), Compound (iv) is 5,11-dihydroindolo-[3,2-b]carbazole (ICZ). In all aspects and embodiments of the invention, preferably the estrogen modulator (B) is Indole-3-carbinole (I3C) and/or 3,3′-diindolylmethane (DIM) or a pharmaceutically acceptable salt, solvate or hydrate thereof, preferably Indole-3-carbinole (I3C) or a pharmaceutically acceptable salt, solvate or hydrate thereof.

More than one estrogen modulator may be present in/as component (B) of the present invention. For example, component (B) may be or may comprise at least one estrogen modulator, e.g. at least two estrogen modulators. If there is more than one estrogen modulator (B), these can be administered simultaneously, separately, or sequentially. The combination of I3C and DIM (or pharmaceutically acceptable salts, solvates or hydrates thereof) is particularly preferred in this regard, as mentioned above. Where there more than one modulator (B), they can be formulated together or separately.

In a particular embodiment, the second component (B) is selected from the following compounds (i′)-(iv'):

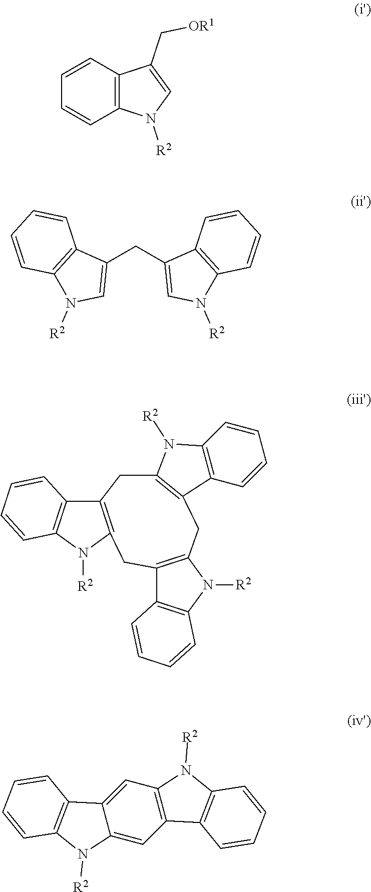

wherein R¹ is selected from H, substituted or unsubstituted hydrocarbyl (e.g. alkyl or aryl), —CO—[C₁-₆alkyl], preferably R¹ is selected from H, C₁-C₆ hydrocarbyl (preferably C₁-C₆ alkyl), —CO—[C₁-₆alkyl], more preferably R¹ is H;
wherein each R² is independently selected from H, substituted or unsubstituted hydrocarbyl (e.g. alkyl or aryl), —CO—[C₁-₆alkyl], preferably R² is selected from H, C₁-C₆ hydrocarbyl (preferably C₁-C₆ alkyl), —CO—[C₁-₆alkyl], more preferably R² is H;
or pharmaceutically acceptable salts or hydrates thereof.

By independently selected is meant for each molecule and within each molecule.

In a particular embodiment, the invention provides a selective estrogen receptor modulator and/or an aromatase inhibitor (A) for use in a method of treating gynecomastia and/or mastalgia,
wherein said method comprises administering said selective estrogen receptor modulator and/or aromatase inhibitor in combination with at least one of the above compounds (i)-(iv) or (i′)-(iv′), or a precursor (e.g. prodrug) or metabolite of compounds (i)-(iv) or (i′)-(iv′).

### Indole-3-Carbinol

Indole-3-carbinol (I3C) is formed from a substance called glucobrassicin found in cruciferous vegetables. By cruciferous vegetables is typically meant broccoli, Brussels sprouts, cabbage, collards, cauliflower, kale, mustard greens, turnips, rutabagas and similar green leaf vegetables. I3C is formed when these vegetables are cut, chewed or cooked. It can also be produced in the laboratory.

In the stomach, I3C molecules undergo acid-catalyzed condensation that generates a number of biologically active I3C oligomers, such as 3,3′-diindolylmethane (DIM), 5,11-dihydroindolo-[3,2-b]carbazole (ICZ), and 5,6,11,12,17,18-hexahydrocyclononal [1,2-b:4,5-b′:7,8-b″]triindole (CT)

Like Tamoxifen and other SERMs, I3C and DIM have been found to modulate the expression and activity of biotransformation enzymes that are involved in the metabolism and elimination of many biologically active compounds, including steroid hormones, drugs, carcinogens, and toxins.

Preclinical studies suggested that anti-estrogenic activities of I3C and DIM might help reduce the risk of hormone-dependent cancers. Although supplementation with I3C and DIM could alter urinary estrogen metabolite profiles in women, the effects of I3C and DIM on breast cancer risk are not known.

Combinations of I3C and the anti-estrogen tamoxifen have been shown to cooperate to inhibit the growth of the estrogen-dependent human MCF-7 breast cancer cell line more effectively than either agent alone (Cover et AI., ‘Indole-3-Carbinol and Tamoxifen Cooperate to Arrest the Cell Cycle of MCF-7 Human Breast Cancer Cells’. J Cancer Research. 1244-1251, 59 (1999)). Several lines of evidence suggest that I3C works through a mechanism distinct from tamoxifen. I3C has been shown to fail to compete with estrogen for estrogen receptor binding, and it specifically down-regulates the expression of CDK6. These results demonstrate that I3C and Tamoxifen work through different signal transduction pathways to suppress the growth of human breast cancer cells.

I3C can be combined with other compounds to improve bioavalability but the active ingredient Indole-3-carbinol when given orally is typically converted to diindolylmethane (see below).

I3C's duration of activity may also be modified by the addition of other molecules that prevent degradation in vivo.

### 3,3′-Diindolylmethane

Indole-3-carbinol (I3C) when given orally is converted to diindolylmethane (DIM) and other oligomers catalyzed by stomach acid (see FIG. 1). This suggests that DIM is the predominant active agent and that I3C is a precursor, ‘pro-drug’ in vivo. However, in cell culture studies carried out in neutral solutions, I3C has been considered fully active.

However, DIM has different biological activity and estrogen suppression to I3C. It seems likely that DIM could also act cooperatively/synergistically to the SERM/AI (e.g. Tamoxifen), alone or in combination with I3C. Cooperativity/synergy may arise from reducing toxicity or enhancing efficacy or reducing resistance to the SERM/AI (e.g. Tamoxifen). In a particular embodiment, therefore, a mixture of I3C and DIM are present in component (B) of the present invention.

DIM can be combined with other compounds to improve bioavalability. For instance, high bioavailability is achieved by administering liquid DIM (oil solution), containing cod liver oil and polysorbate compared to non-formulated crystalline DIM. DIM's duration of activity may also be modified by the addition of other molecules that prevent degradation in vivo. DIM can be combined with d-alpha-tocopheryl polyethylene glycol-1000 succinate to make it more absorbable by the gut. In a particular embodiment, therefore, DIM is administered as a liquid (oil solution), wherein the liquid contains cod liver oil and polysorbate. In a particular embodiment, DIM is combined with d-alpha-tocopheryl polyethylene glycol-1000 succinate.

### Food/vegetable supplement (e.g. Cruciferous vegetable supplement)

In a particular embodiment, component (B) is a food supplement, e.g. a vegetable supplement, e.g. a cruciferous vegetable supplement.

In a particular embodiment, the estrogen modulator is a glucobrassin derivative. By ‘derivative’ is typically meant in this context a compound that is formed upon cutting, chewing, extracting or cooking these cruciferous vegetables. Typically, the derivatives for use in combination with the SERM and/or AI are compounds formed from the hydrolysis of glucobrassin.

In a further aspect of the invention, the invention provides the use of glucobrassin per se, or derivatives such as 1-Methoxyglucobrassicin (neoglucobrassicin), 4-Hydroxyglucobrassicin, 4-Methoxyglucobrassicin, 1,4-Dimethoxyglucobrassicin, 1-Sulfoglucobrassicin, 6′-Isoferuloylglucobrassicin, as component (B) in combination with a SERM and/or an AI. In a particular embodiment, therefore, the invention provides for the combination of a SERM and/or an AI, in combination with glucobrassin or one of these derivatives, for use in the treatment of gynecomastia and/or mastalgia.

In a particular embodiment, the estrogen modulator is derived from cruciferous vegetables. Cruciferous vegetables, or brassic vegetables, encompass the following: broccoli, Brussels sprouts, cabbage, collards, cauliflower, kale, mustard greens, turnips, rutabagas and similar green leaf vegetables.

In a particular embodiment, the present invention concerns a selective estrogen receptor modulator and/or an aromatase inhibitor (A) (preferably tamoxifen or a pharmaceutically acceptable salt, solvate or hydrate thereof) for use in a method of preventing or treating gynecomastia and/or mastalgia,
wherein said method comprises administering said selective estrogen receptor modulator and/or aromatase inhibitor in combination with
(B): a cruciferous vegetable supplement or extract (e.g. I3C, or I3C and DIM, or pharmaceutically acceptable salts, solvates or hydrates thereof).

By supplement or extract is meant both synthetically and naturally produced compounds. A cruciferous vegetable supplement/extract or a brassic vegetable supplement/extract can encompass compounds found in these vegetables (both synthetic or extracted), synthetic versions thereof, as well as compounds which may not be present in the vegetables per se but are compounds found in cruciferous/brassic vegetable material, i.e. after it has been chopped, cut, extracted or treated in some way.

There is no clinical data attesting to herbal supplements use in gynecomastia or mastalgia although certain supplements have been shown to have potential as estrogen modulator agents, typically in the context of alternative hormone therapies to treat cancer.

Other food extracts that have reputedly estrogen modulator effects are wild nettle root, chrysin, soy, turmeric, maca, flavonoids and grape seed extract. In a particular embodiment, the present invention concerns the combination of the following in the treatment or prevention of gynecomastia and/or mastalgia:
(A): A selective estrogen receptor modulator and/or an aromatase inhibitor (preferably tamoxifen or a pharmaceutically acceptable salt, solvate or hydrate thereof);
(B) wild nettle root, chrysin, soy, turmeric, maca, flavonoids and grape seed extract.

### Synergistic/Cooperative Combinations

Tamoxifen (TAM) is known to have a dual mechanism of action: (1) to compete with 17β-estradiol (E2) at the receptor site and to block the promotional role of E2 in tissues. This is demonstrated by experiments in estrogen receptor knockout mice that display grossly impaired breast ductal development.

In humans, given the predominant use of Tamoxifen in female breast cancer the vast majority of the clinical and pharmacological data is based in this setting. Although Tamoxifen generally acts as an estrogen receptor antagonist in breast cancer cells, in certain other cell types, tamoxifen can act as an estrogen receptor agonist. Several mechanisms are proposed for the modulation of cellular growth by tamoxifen, including modulation of growth factor signalling, regulation of the cell-cycle machinery and down-regulation of oncogenes. Whilst tamoxifen has been shown to decrease the activity of the estrogen receptor it does not have an antiproliferative effect on estrogen receptor cell lines. This is an important characteristic for this application as I3C (or other estrogen modulators) can suppress the growth of cells regardless of estrogen receptor status.

Given the known idiosyncratic features of the growth suppression cascades induced by I3C and tamoxifen, Cover et al. (1999) demonstrated that a combination of tamoxifen and I3C displayed a more effective growth suppression response, a more stringent inhibition of CDK2 specific activity, and more endogenous Rb phosphorylation than either compound alone. The authors recommend exploring I3C and tamoxifen as a potential combinatorial therapy to control estrogen-responsive breast cancers. The authors further suggested that I3C, in combination with tamoxifen, could overcome some of the drawbacks of tamoxifen therapy while capitalizing on the positive effects of this proven therapy. Lower doses and/or pulses of tamoxifen are two of the proposed methods of circumventing tamoxifen resistance. In this regard, their results showed that lower doses of tamoxifen and I3C inhibited MCF-7 cell growth and CDK2-specific activity to the same extent as higher doses of either agent added individually. In principle, this response could be exploited to circumvent acquired drug resistance to sustained high doses of tamoxifen. Alternatively, patients could conceivably receive intermittent pulses of tamoxifen while undergoing I3C treatment. I3C has been shown to reduce the formation of both spontaneous and carcinogen-induced mammary tumors in rodents with no apparent side effects. Human subjects who ingested I3C also had no side effects.

As mentioned above, the estrogen modulator (B) can affect estrogen production via a different biochemical pathway to the SERM and/or AI (A). Indeed, Tamoxifen and I3C operate on the estrogen pathway through different mechanisms of action and are therefore cooperative in their action on gynecomastia/mastalgia. (see Table 1).

TABLE 1
Mechanisms of action of Tamoxifen (TAM)
and I3C on the estrogen pathway

| Mechanism | TAM | I3C |
|---|---|---|
| Competes with estrogen | yes | no |
| Triggers degradation of ERα | no | yes |
| Inhibits estrogen synthesis | no | yes |
| Enhances 2HE1 excretion | no | yes |
| Lowers circulating estrogen | no | yes |

Surprisingly, the present inventor has found that the combination of components (A) and (B) offers positive results in gynecomastia and/or mastalgia treatment.

### Formulation

The components/compounds (A) and (B) of the invention are preferably formulated as pharmaceutically acceptable compositions. The phrase “pharmaceutically acceptable”, as used in connection with compositions of the invention, refers to molecular entities and other ingredients of such compositions that are physiologically tolerable and do not typically produce untoward reactions when administered to a mammal (e.g. human). Preferably, as used herein, the term “pharmaceutically acceptable” means approved by a regulatory agency of the Federal or a state government or listed in the U.S. Pharmacopoeia or other generally recognized pharmacopoeia for use in mammals, and more particularly in humans.

It will be appreciated that components/compounds (A) and (B) of the invention can be administered in salt, solvate, hydrate prodrug or ester form, especially salt form. The invention therefore also provides pharmaceutically acceptable salts, esters, solvates, or prodrugs of the compounds described herein, whether the selective estrogen receptor modulator and/or an aromatase inhibitor (A), or the estrogen modulator (B), in particular pharmaceutically-acceptable salts thereof. We discuss suitable salt, solvate, prodrug or ester forms below. Typically, a pharmaceutically acceptable salt may be readily prepared by using a desired acid.

The components/compounds of the invention can be administered in salt, solvate, hydrate prodrug or ester form, especially salt form. Typically, a pharmaceutical acceptable salt may be readily prepared by using a desired acid. The salt may precipitate from solution and be collected by filtration or may be recovered by evaporation of the solvent. For example, an aqueous solution of an acid such as hydrochloric acid may be added to an aqueous suspension of a compound and the resulting mixture evaporated to dryness (lyophilised) to obtain the acid addition salt as a solid. Alternatively, a compound of the invention may be dissolved in a suitable solvent, for example an alcohol such as isopropanol, and the acid may be added in the same solvent or another suitable solvent. The resulting acid addition salt may then be precipitated directly, or by addition of a less polar solvent such as diisopropyl ether or hexane, and isolated by filtration.

Suitable addition salts are formed from inorganic or organic acids which form non-toxic salts and examples are hydrochloride, hydrobromide, hydroiodide, sulphate, bisulphate, nitrate, phosphate, hydrogen phosphate, acetate, trifluoroacetate, maleate, malate, fumarate, lactate, tartrate, citrate, formate, gluconate, succinate, pyruvate, oxalate, oxaloacetate, trifluoroacetate, saccharate, benzoate, alkyl or aryl sulphonates (eg methanesulphonate, ethanesulphonate, benzenesulphonate or p-toluenesulphonate) and isethionate. Representative examples include trifluoroacetate and formate salts, for example the bis or tris trifluoroacetate salts and the mono or diformate salts, in particular the tris or bis trifluoroacetate salt and the monoformate salt. A particularly preferred salt of Tamoxifen is its citrate salt. In a particular embodiment, therefore, Tamoxifen, if present, if administered as Tamoxifen citrate.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of the compounds of the invention are within the scope of the invention. The salts of the compounds/components (A) and (B) may form solvates (e.g. hydrates) and the invention also includes all such solvates.

The term “prodrug” as used herein means a compound which is converted within the body, e.g. by hydrolysis in the blood, into its active form that has medical effects.

In a particular embodiment, the present invention concerns the combination of the following in the treatment of gynecomastia and/or mastalgia:
(A): a pharmaceutical composition comprising a selective estrogen receptor modulator and/or an aromatase inhibitor (preferably tamoxifen or a pharmaceutically acceptable salt, solvate or hydrate thereof);
(B) a pharmaceutical composition comprising an estrogen modulator, wherein said estrogen modulator is different to said selective estrogen receptor modulator.

While it is possible that, for use in the methods of the invention, a compound of the invention (e.g. the SERM/AI (A) or the estrogen modulator (B)) may be administered as the bulk substance, it is preferable to present the active ingredient in a pharmaceutical formulation, for example, wherein the agent is in admixture with a pharmaceutically acceptable excipient or carrier selected with regard to the intended route of administration and standard pharmaceutical practice. All of the discussion about use in the treatment or prevention of gynecomastia and/or mastalgia thus also applies to the formulations of the invention. In a particular embodiment, therefore, the invention provides a pharmaceutical composition comprising (A) and (B) and at least one excipient, for use in the treatment of gynecomastia and/or mastalgia.

In a particular embodiment, the invention provides a pharmaceutical composition (A) comprising a selective estrogen receptor modulator and/or an aromatase inhibitor for use in a method of preventing or treating gynecomastia and/or mastalgia,
wherein said method comprises administering said pharmaceutical composition (A) in combination with
(B) a pharmaceutical composition (B) comprising an estrogen modulator, wherein said estrogen modulator is different to said selective estrogen receptor modulator.

Such considerations apply to all other compounds/compositions for use, uses, methods etc. of the invention.

The term “excipient” refers to a diluent, carrier, and/or vehicle with which an active compound is administered. Any pharmaceutical compositions (whether for components (A) or (B)) discussed herein may contain combinations of more than one excipient or carrier. Such pharmaceutical excipients or carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like.

Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18th Edition. The choice of pharmaceutical carrier can be selected with regard to the intended route of administration and standard pharmaceutical practice. The pharmaceutical compositions may comprise as, in addition to, the excipient any suitable binder(s), lubricant(s), suspending agent(s), coating agent(s), and/or solubilizing agent(s). Particularly preferred for the present invention are carriers suitable for immediate-release, i.e., release of most or all of the active ingredient over a short period of time, such as 60 minutes or less, and make rapid absorption of the drug possible.

It will be appreciated that pharmaceutical compositions (whether for (A) or (B)) for use in accordance with the present invention may be in the form of oral, parenteral, transdermal, inhalation, sublingual, topical, implant, nasal, or enterally administered (or other mucosally administered) suspensions, capsules or tablets, gel, liquid preparations, skin patches, injections and infusions, which may be formulated in conventional manner using one or more pharmaceutically acceptable carriers or excipients. Preferably both components (A) and (B) in the present invention are administered in the form of capsules or tablets. In a further embodiment, components (A) and/or (B) are administered as a topical formulation (e.g. cream, skin patch, gel etc.).

Also considered are key active ingredients, being modified to improve bioavailability or delivered in media (for instance gels versus tablets) that have a similar mode of action on estrogen but differing bioavailabilities, duration, efficacy, toxicity or tolerance effects.

There may be different composition/formulation requirements depending on the different delivery systems. Likewise, if the composition comprises more than one active component, then those components may be administered by the same or different routes.

The pharmaceutical formulations of the present invention can be liquids that are suitable for oral, mucosal and/or parenteral administration, for example, drops, syrups, solutions, injectable solutions that are ready for use or are prepared by the dilution of a freeze-dried product but are preferably solid or semisolid as tablets, capsules, granules, powders, pellets, pessaries, suppositories, creams, salves, gels, ointments; or solutions, suspensions, emulsions, or other forms suitable for administration by the transdermal route or by inhalation.

Tamoxifen is mainly available in two forms: a pill taken once a day (brand name: Nolvadex) or a liquid form (brand name: Soltamox). In a particular embodiment, therefore, component (A) (i.e. the SERM and/or AI) is administered as a pill/capsule/tablet or as a liquid (i.e. a solution/suspension).

The compounds of the invention can be administered for immediate-, delayed-, modified-, sustained-, pulsed- or controlled-release applications.

In one aspect, oral compositions are slow, delayed or positioned release (e.g., enteric especially colonic release) tablets or capsules. This release profile can be achieved without limitation by use of a coating resistant to conditions within the stomach but releasing the contents in the colon or other portion of the GI tract wherein a site has been identified or a delayed release can be achieved by a coating that is simply slow to disintegrate or the two (delayed and positioned release) profiles can be combined in a single formulation by choice of one or more appropriate coatings and other excipients. Such formulations constitute a further feature of the present invention.

Pharmaceutical compositions can be prepared by mixing a therapeutically effective amount of the active substance with a pharmaceutically acceptable carrier that can have different forms, depending on the way of administration. Typically composition components include one or more of binders, fillers, lubricants, odorants, dyes, sweeteners, surfactants, preservatives, stabilizers and antioxidants.

The pharmaceutical compositions of the invention (i.e. for (A) and (B)) may contain from 0.01 to 99% weight-per volume of the active material (e.g. a pharmaceutical composition comprising a SERM and/or AI may contain from 0.01 to 99% weight SERM and/or AI, a pharmaceutical composition comprising an estrogen modulator may contain from 0.01 to 99% weight estrogen modulator). The total therapeutic doses (i.e. both SERM/AI (A) and estrogen modulator (B) combined) will generally be between about 10 and 2000 mg/day and preferably between about 30 and 1500 mg/day. Other ranges may be used, including, for example, 50-500 mg/day, 50-300 mg/day, 100-200 mg/day.

Administration may be once a day, twice a day, or more often, and may be decreased during a maintenance phase of the disease or disorder, e.g. once every second or third day instead of every day or twice a day. The dose and the administration frequency will depend on the clinical signs, which confirm maintenance of the remission phase, with the reduction or absence of at least one or more preferably more than one clinical signs of the acute phase known to the person skilled in the art.

It is within the scope of the invention for a compound as described herein to be administered in combination with another pharmaceutical, e.g. another drug with known efficacy against gynecomastia and/or mastalgia. ‘In combination’ here means in parallel; the other agents may be administered before, during, or after administration of the compound/formulation of the invention.

### Application

Components (A) and (B) of the invention, in all aspects of the invention, may be administered simultaneously, separately, or sequentially, i.e. the selective estrogen receptor modulator and/or an aromatase inhibitor (A) may be administered with estrogen modulator (B) simultaneously, separately, or sequentially. This applies to all embodiments/aspects of the invention, whether compositions/compounds for use, kits for use, methods, uses, etc.

The components (A) and (B) of the invention are used in combination therapy. Broadly speaking, the combination therapy makes use of two components: (A) and (B). Ingredient/component (A) is or comprises a selective estrogen receptor modulator and/or an aromatase inhibitor. Ingredient/component (B) is or comprises an estrogen modulator, wherein said estrogen modulator is different to said selective estrogen receptor modulator. ‘In combination’ here means in parallel; component (B) may be administered before, during, or after administration of component (A). Component (B) may be formulated together with component (A), or separately. In a particular embodiment, the invention provides a composition (e.g. a pharmaceutical composition/formulation) comprising:
(A) a selective estrogen receptor modulator and/or an aromatase inhibitor; and
(B) an estrogen modulator,
for use in the treatment or prevention of gynecomastia and/or mastalgia.

The compounds of the invention may be used in combination with other compounds with known efficacy against gynecomastia and/or mastalgia.

The invention also concerns the combination of (A) and (B) [e.g. a kit comprising (A) and (B) or a composition comprising (A) and (B))] per se. In a particular embodiment, therefore, the invention provides a composition (e.g. a pharmaceutical formulation) comprising:
(A) a selective estrogen receptor modulator and/or an aromatase inhibitor; and
(B) an estrogen modulator, wherein said estrogen modulator is different to said selective estrogen receptor modulator.

In a further embodiment, the invention provides a kit (e.g. a pharmaceutical kit of parts) comprising:
(A) a first composition comprising or consisting of a selective estrogen receptor modulator and/or an aromatase inhibitor; and
(B) a second composition comprising or consisting of an estrogen modulator, wherein said estrogen modulator is different to said selective estrogen receptor modulator.

### Non-Therapeutic Use

Should any use described herein be considered to be non-therapeutic, then the present invention provides also the use of components (A) and (B) in the reduction of breast size in a subject, e.g. human, e.g. a male human. Said human, e.g. male human, may be an adult or a child (i.e. under 18 years of age). The invention provides also a method of reducing the breast size in a human, e.g. a male human, comprising administering components (A) and (B) of the invention. Said administration can again be simultaneous, separate, or sequential. Said use/method can be non-therapeutic and/or cosmetic.

### Gynecomastia and/or Mastalgia Treatment

The compounds and combinations of the invention are proposed for use in the treatment or prevention of gynecomastia and/or mastalgia. By treating or treatment is meant at least one of:
(i). preventing or delaying the appearance of clinical symptoms of the disease developing in a mammal;
(ii). inhibiting the disease i.e. arresting, reducing or delaying the development of the disease or a relapse thereof or at least one clinical or subclinical symptom thereof, or
(iii). relieving or attenuating one or more of the clinical or subclinical symptoms of the disease.

Typically, treating gynecomastia refers to reducing the size of breasts in subjects who suffer from enlarged breasts as a result of gynecomastia. Typically, treating mastalgia refers to reducing the pain of breasts in subjects who suffer from enlarged breasts as a result of hormonal imbalances.

Mastalgia may herein refer to gynecomastia-related mastalgia. The compounds and combinations of the invention may be used for gynecomastia prevention/treatment, mastalgia prevention/treatment, or both. Typically, for mastalgia, the compounds or combinations of the invention are used in treatment as opposed to prevention.

The benefit to a subject to be treated is either statistically significant or at least perceptible to the patient or to the physician. In general a skilled man can appreciate when “treatment” occurs.

The word prevention is used herein to cover prophylactic treatment, i.e. treating subjects who are at risk of developing gynecomastia and/or mastalgia.

Exercise (e.g. press-ups) is (are) not known to affect gynecomastia but can help increase muscle mass in the chest area.

The compounds or combinations of the invention can be used on any animal subject, in particular a mammal and more particularly to a human or an animal serving as a model for a disease (e.g. mouse, monkey, etc.), preferably a human, preferably a male human. The human may also be a woman (including transgender woman). The human may be an adult or a child (i.e. under 18 years of age).

An “effective dose” means the amount of a compound that, when administered to an animal for treating a state, disorder or condition, is sufficient to effect such treatment. The “effective dose” will vary depending on the compound, the severity of the disorder/disease and the age, weight, physical condition and responsiveness of the subject to be treated and will be ultimately at the discretion of the attendant doctor.

In all aspects or embodiments of the invention (compounds for use, uses, methods etc.), the following combination of components is highly preferred:
(A) Tamoxifen, or a pharmaceutically acceptable salt, solvate or hydrate thereof; and
(B) Indole-3-carbinole (I3C) and/or 3,3′-diindolylmethane (DIM), or pharmaceutically acceptable salts, solvates or hydrates thereof.

In a particular embodiment, the invention therefore provides Tamoxifen, or a pharmaceutically acceptable salt, solvate or hydrate thereof, for use in a method of preventing or treating gynecomastia and/or mastalgia, wherein said method comprises administering said Tamoxifen (or pharmaceutically acceptable salt, solvate or hydrate thereof) in combination with Indole-3-carbinole (I3C) and/or 3,3′-diindolylmethane (DIM), or pharmaceutically acceptable salts, solvates or hydrates thereof.

Clinical trial data has shown that daily dosing of 10 mg or 20 mg Tamoxifen (TAM) is promising for the treatment of gynecomastia and/or mastalgia. These daily dosages represent typical embodiments for the SERM and/or AI. The duration of treatment typically ranges from 3 to 9 months.

I3C is typically taken as a daily food supplement in doses ranging from 100 mg to 500 mg per day. DIM is typically taken as a daily supplement in doses raging from 100 mg to 1000 mg per day. Therapeutic doses in early stage clinical trials are 2-3× that given as part of a daily supplement regimen. A suitable daily dosage for I3C is therefore 100 mg to 1500 mg/day. A suitable dosage range for DIM is therefore 100 mg to 3000 mg per day.

In a particular embodiment, the selective estrogen receptor modulator and/or aromatase inhibitor (A) is administered at a dosage of 1-1000 mg/day, preferably 1-100 mg/day, preferably 1-50 mg/day, preferably 5-50 mg/day, preferably 10-40 mg/day. A particularly suitable dosage range for (A) is 1-20 mg/day.

In a particular embodiment, the estrogen modulator (B) is administered at a dosage of 1-5000 mg/day, preferably 1-2500 mg/day, preferably 1-1000 mg/day, preferably 50-750 mg/day, preferably 100-400 mg/day.

If there are two or more estrogen modulators (B), the dosages described above refer to a combined dose. The same applies if there is both a SERM and an AI as component (A), or two or more SERMs.

The following treatment protocols, each taken once or twice daily in a separate or combined tablet form, e.g. over 1-12 months, e.g. over 3, 6 or 9 months, are therefore considered to be of particular interest for the present invention, and represent particular embodiments thereof:
1) TAM 10 mg+I3C 200 mg.
2) TAM 10 mg+I3C 100 mg+DIM 100 mg.
3) TAM 10 mg+DIM 200 mg.
4) TAM 20 mg+DIM 200 mg.
5) TAM 20 mg+I3C 200 mg+DIM 200 mg.
6) TAM 20 mg+I3C 500 mg+DIM 500 mg.
7) TAM 20 mg+DIM 1000 mg.
8) TAM 20 mg+DIM 1000 mg+I3C 500 mg.
9) TAM 20 mg+I3C 500 mg.
10) TAM 5 mg+I3C 200 mg.
11) TAM 5 mg+I3C 400 mg.
12) TAM 5 mg+I3C 500 mg.
13) TAM 5 mg+I3C 1000 mg.
14) TAM 2 mg+I3C 200 mg.
15) TAM 2 mg+I3C 400 mg.
16) TAM 2 mg+I3C 500 mg.
17) TAM 2 mg+I3C 1000 mg.
18) TAM 1 mg+I3C 200 mg.
19) TAM 1 mg+I3C 400 mg.
20) TAM 1 mg+I3C 500 mg.
21) TAM 1 mg+I3C 1000 mg.

Treatment protocols with dosages within +/−50% of the above values are considered herein and also form particular embodiments of the invention. The dosage of TAM 5 mg+I3C 200 mg is particularly preferred.

### BRIEF DESCRIPTION OF FIGURES

FIG. 1 shows the acid-catalyzed condensation of indole-3-carbinole (I3C) to generate the biologically active I3C oligomers (ii) 3,3′-diindolylmethane (DIM), (iii) 5,6,11,12,17,18-hexahydrocyclononal [1,2-b:4,5-b′:7,8-b″]triindole (CT) and (iv) 5,11-dihydroindolo-[3,2-b]carbazole (ICZ).

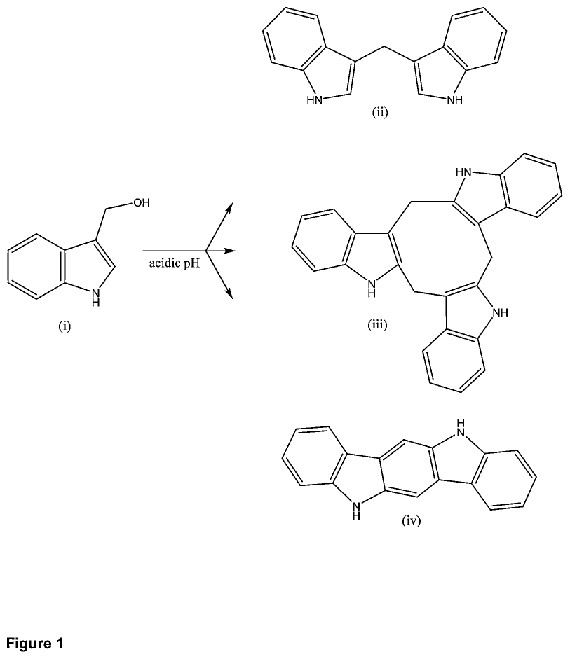

FIG. 2 shows anti-estrogenic actions of indole-3-carbinole (I3C) and DIM 3,3′-diindolylmethane (DIM) (AhR=hydrocarbon receptor; CYP=cytochrome P450, ERα=estrogen receptor alpha, ESR1=gene encoding ERα) [reference 7].

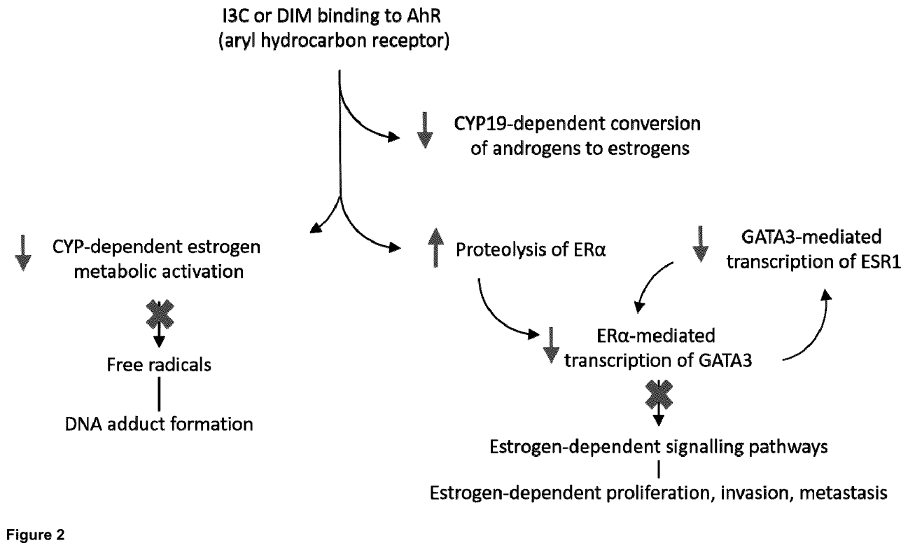

FIG. 3 shows photographs at week 1 of treatment with daily dose of 5 mg tamoxifen along with a separate tablet of 200 mg I3C.

FIG. 4 shows photographs at week 2 of treatment with daily dose of 5 mg tamoxifen along with a separate tablet of 200 mg I3C.

FIG. 5 shows photographs at week 3 of treatment with daily dose of 5 mg tamoxifen along with a separate tablet of 200 mg I3C.

FIG. 6 shows photographs at week 4 of treatment with daily dose of 5 mg tamoxifen along with a separate tablet of 200 mg I3C.

FIG. 7 shows photographs at week 5 of treatment with daily dose of 5 mg tamoxifen along with a separate tablet of 200 mg I3C.

FIG. 8 shows photographs at week 10 of treatment with daily dose of 5 mg tamoxifen along with a separate tablet of 200 mg I3C.

FIG. 9 shows photographs at week 11 of treatment with daily dose of 5 mg tamoxifen along with a separate tablet of 200 mg I3C.

FIG. 10 shows photographs at week 12 of treatment with daily dose of 5 mg tamoxifen along with a separate tablet of 200 mg I3C.

### EXAMPLE

### Introduction

A male subject suffering from gynecomastia was administered with a combination of Tamoxifen and I3C. The purpose of the case study trial was to gauge any harmful effects of the combination along with any positive efficacy.

### Method

(I) Over a three-month period the subject took a daily dose of 5 mg tamoxifen along with a separate tablet of 200 mg I3C in the morning before breakfast. This regimen was continued for three months. The subject also increased press up exercises from 5 per day to 30 per day by the end of the programme.

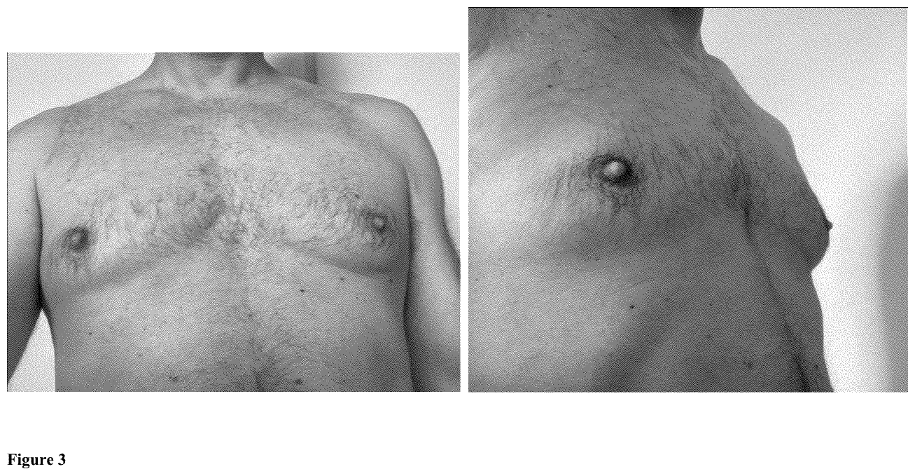

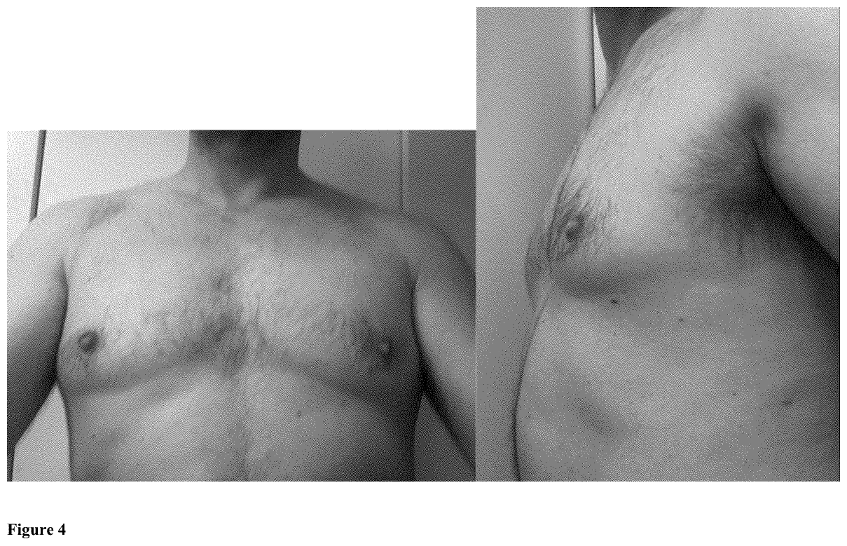

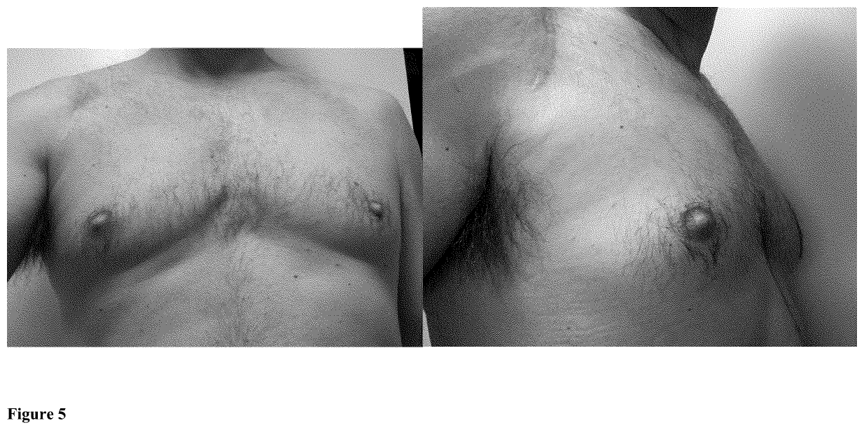

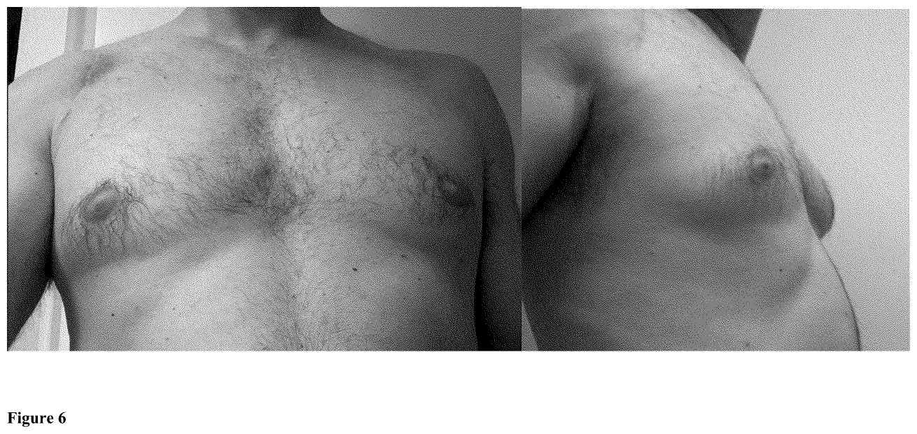

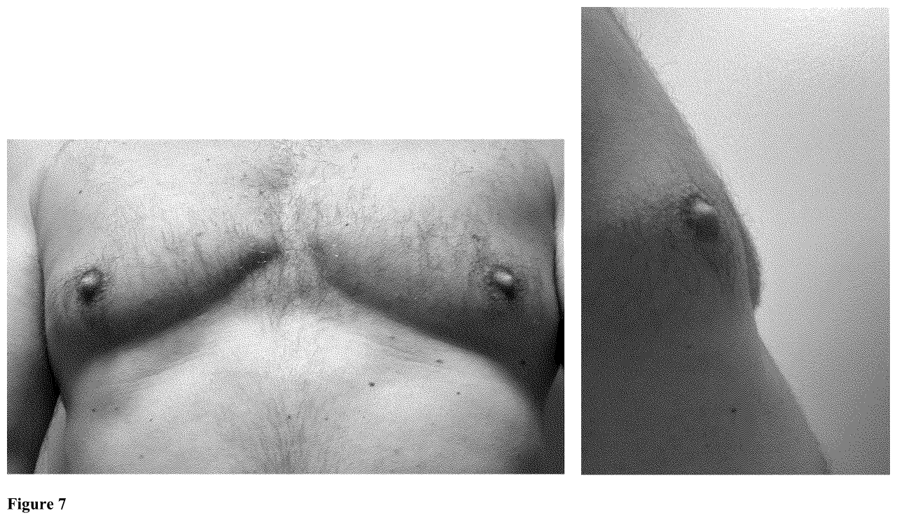

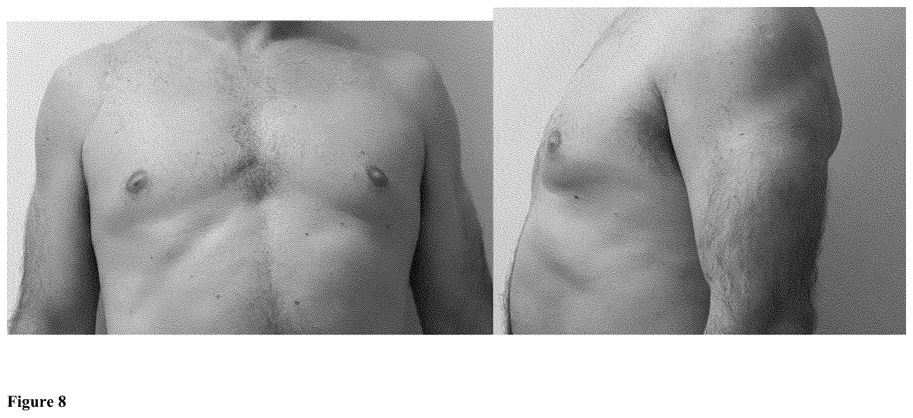

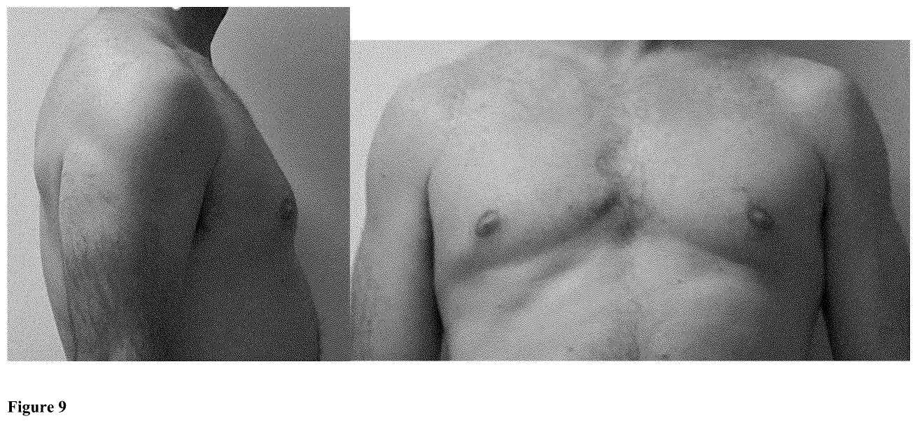

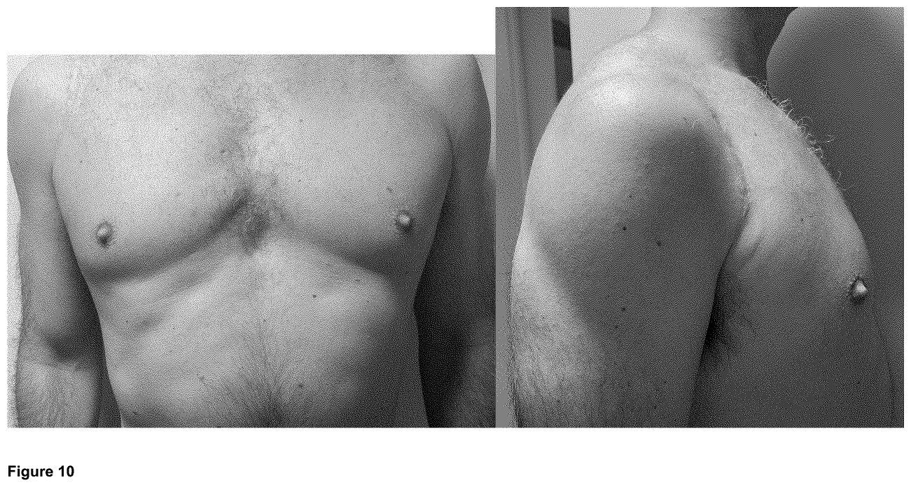

Reduction in breast size is evidenced by photographs taken over the course of the experiment. No mastalgia was reported. A photographic record spanning the three-month period (up to week 12) is shown in FIGS. 3-10. Signs of gynecomastia improvement, as indicated by smaller breasts, began after three weeks and increased over the course of the treatment. There were no noticeable side-effects of treatment. Indeed, Tamoxifen and I3C are known to cause headaches, nausea, vomiting, weight gain, impotence and loss of libido. Surprisingly, despite the concomitant treatment, the subject experienced efficacy with no side effects associated with the trial.

(II) Over a three-month period the subject took a daily dose of 20 mg tamoxifen along with a separate tablet of 200 mg I3C in the morning before breakfast. This regimen was continued for three months. The subject also increased press up exercises from 5 per day to 30 per day by the end of the programme.

### BIBLIOGRAPHY

1. Scurr J, Hedger W, Morris P, Brown N. The prevalence, severity, and impact of breast pain in the general population. Breast J. 2014;20(5):508-513. doi:10.1111/tbj.12305
2. Olawaiye A, Withiam-Leitch M, Danakas G, Kahn K. Mastalgia: a review of management. J Reprod Med. 2005;50(12):933-939.
3. Hafiz SP, Barnes NLP, Kirwan CC. Clinical management of idiopathic mastalgia: a systematic review. J Prim Health Care. 2018;10(4):312-323. doi:10.1071/HC18026
4. Smetnik, Antonina & Smetnik, Smetnik & Kiselev, Kiselev. (2017). Experience with indole-3-carbinol used to treat diseases of the breast and to prevent breast cancer. Akusherstvo i ginekologiia. 2_2017. 106-112. 10.18565/aig.2017.2.106-12.
5. Thomson CA, Chow HHS, Wertheim BC, et al. A randomized, placebo-controlled trial of diindolylmethane for breast cancer biomarker modulation in patients taking tamoxifen. Breast Cancer Res Treat. 2017;165(1):97-107. doi:10.1007/s10549-017-4292-7
6. Mannu GS, Sudul M, Bettencourt-Silva JH, Tsoti SM, Cunnick G, Ahmed SF. Role of tamoxifen in idiopathic gynecomastia: A 10-year prospective cohort study. Breast J. 2018 November;24(6):1043-1045. doi:10.1111/tbj.13080. Epub 2018 Aug. 5. PMID: 30079473.
7. Investigation and Management of Gynaecomastia in Primary & Secondary Care. Association of Breast Surgery Summary Statement. https://associationofbreastsurgery.org.uk/media/65097/abs-summary-statement-gynecomastia-2019.pdf
8. Johnson RE, Murad MH. Gynecomastia: pathophysiology, evaluation, and management. Mayo Clin Proc. 2009;84(11):1010-1015. doi:10.1016/S0025-6196(11)60671-X
9. Medication and Gynecomastia. Gynecomastia website. https://www.gynecomastia.org/gynecomastia-101/medication-and-gynecomastia
10. Woo HI, Lee SK, Kim J, et al. Variations in plasma concentrations of tamoxifen metabolites and the effects of genetic polymorphisms on tamoxifen metabolism in Korean patients with breast cancer. Oncotarget. 2017;8(59):100296-100311. Published 2017 Nov. 1. doi:10.18632/oncotarget.22220
11. de Ronde, W., de Jong, F.H. Aromatase inhibitors in men: effects and therapeutic options. Reprod Biol Endocrinol 9, 93 (2011). https://doi.org/10.1186/1477-7827-9-93
12. “Indole-3-Carbinol” website of Oregon State University. Indole-3-Carbinol|Linus Pauling Institute| Oregon State University: https://lpi.oregonstate.edu/mic/dietary-factors/phytochemicals/indole-3-carbinol
13. Swerdloff RS, Ng CM. Gynecomastia: Etiology, Diagnosis, and Treatment. [Updated 2019 Jul. 7]. In: Feingold KR, Anawalt B, Boyce A, et al., editors. Endotext [Internet]. South Dartmouth (MA): MDText.com, Inc.; 2000-. Available from: https://www.ncbi.nlm.nih.gov/books/NBK279105/
14. Harold E. Carlson, Approach to the Patient with Gynecomastia, The Journal of Clinical Endocrinology & Metabolism, Volume 96, Issue 1, 1 Jan. 2011, Pages 15-21, https://doi.org/10.1210/jc.2010.1720
15. Parker LN, Gray DR, Lai MK, Levin ER. Treatment of gynecomastia with tamoxifen: a double-blind crossover study. Metabolism. 1986 August;35(8):705-8. doi:10.1016/0026-0495(86)90237-4. PMID: 3526085.
16. Braunstaun et al. Patient education: Gynecomastia (breast enlargement in men) (Beyond the Basics).
17. Enlarged Breasts in Men (gynecomastia). Mayo Clinic website. https://www.mayoclinic.org/diseases-conditions/gynecomastia/diagnosis-treatment/drc-20351799
18. Kunath, F., Keck, B., Antes, G. et al. Tamoxifen for the management of breast events induced by non-steroidal antiandrogens in patients with prostate cancer: a systematic review. BMC Med 10, 96 (2012). https://doi.org/10.1186/1741-7015-10-96
19. de Ronde W, de Jong FH. Aromatase inhibitors in men: effects and therapeutic options. Reprod Biol Endocrinol. 2011;9:93. Published 2011 Jun. 21. doi:10.1186/1477-7827-9-93
20. Carlson HE. Approach to the patient with gynecomastia. J Clin Endocrinol Metab. 2011 January;96(1):15-21. doi:10.1210/jcem.96.9.zeg15a. PMID: 21209041.
21. Cover CM, Hsieh SJ, Cram EJ, Hong C, Riby JE, Bjeldanes LF, Firestone GL. Indole-3-carbinol and tamoxifen cooperate to arrest the cell cycle of MCF-7 human breast cancer cells. Cancer Res. 1999 Mar. 15;59(6):1244-51. PMID: 10096555.
22. Malejka-Giganti D, Parkin DR, Bennett KK, et al. Suppression of mammary gland carcinogenesis by post-initiation treatment of rats with tamoxifen or indole-3-carbinol or their combination. Eur J Cancer Prev. 2007;16(2):130-141. doi:10.1097/01.cej.0000228401.14988.50
23. Balunas MJ, Su B, Brueggemeier RW, Kinghorn AD. Natural products as aromatase inhibitors. Anticancer Agents Med Chem. 2008 August;8(6):646-82. PMID: 18690828; PMCID: PMC3074486.
24. Quirke VM. Tamoxifen from Failed Contraceptive Pill to Best-Selling Breast Cancer Medicine: A Case-Study in Pharmaceutical Innovation. Front Pharmacol. 2017;8:620. Published 2017 Sep. 12. doi:10.3389/fphar.2017.00620
25. Auborn KJ, Fan S, Rosen EM, Goodwin L, Chandraskaren A, Williams DE, Chen D, Carter TH. Indole-3-carbinol is a negative regulator of estrogen. J Nutr. 2003 July;133(7 Suppl):2470S-2475S. doi:10.1093/jn/133.7.2470s. PMID: 12840226.
26. Narula, H., Carlson, H. Gynaecomastia—pathophysiology, diagnosis and treatment. Nat Rev Endocrinol 10, 684-698 (2014). https://doi.org/10.1038/nrendo.2014.139
27. Harding and Bonsall. Gynecomastia. Patient Information website. Gynaecomastia. Information about Gynaecomastia|Patient: https://patient.info/doctor/gynaecomastia.
28. Wibowo E, Pollock PA, Hollis N, Wassersug RJ. Tamoxifen in men: a review of adverse events. Andrology. 2016 September;4(5):776-88. doi:10.1111/andr.12197. Epub 2016 May 6. PMID: 27152880.
29. Yao Jingwei, Deng Kun, Huang Jialu, Zeng Ruimin, Zuo Jianhong. Progress in the Understanding of the Mechanism of Tamoxifen Resistance in Breast Cancer. Frontiers in Pharmacology. 11, 1848 (2020).
30. Pemmaraju N, Munsell MF, Hortobagyi GN, Giordano SH. Retrospective review of male breast cancer patients: analysis of tamoxifen-related side-effects. Ann Oncol. 2012 June;23(6):1471-4. doi:10.1093/annonc/mdr459. Epub 2011 Nov. 15. PMID: 22085764; PMCID: PMC3493137

1. A method of preventing or treating gynecomastia and/or mastalgia in a subject in need thereof,
comprising administering to the subject (A) a selective estrogen receptor modulator and/or aromatase inhibitor in combination with
(B) an estrogen modulator, wherein said estrogen modulator is different to the selective estrogen receptor modulator.

2. The method as claimed in claim 1,
comprising administering (A) said selective estrogen receptor modulator and/or aromatase inhibitor in combination with
(B) a supplement comprising an estrogen modulator.

3. The method of claim 1 wherein the selective estrogen receptor modulator and/or aromatase inhibitor (A) is a selective estrogen receptor modulator.

4. The method of claim 1 wherein the selective estrogen receptor modulator (A) is a triphenylethylene-type selective estrogen receptor modulator.

5. The method of claim 1 wherein the selective estrogen receptor modulator (A) is selected from Tamoxifen, Raloxifene and Clomifene, or pharmaceutically acceptable salts, solvates or hydrates thereof.

6. The method of claim 1 wherein the aromatase inhibitor is selected from testolactone, anastrozole and letrozole, or pharmaceutically acceptable salts, solvates or hydrates thereof.

7. The method of claim 1 wherein the estrogen modulator (B) is selected from Indole-3-carbinole (I3C), 3,3′-diindolylmethane (DIM), 5,11-dihydroindolo-[3,2-b]carbazole (ICZ), and/or and 5,6, 11,12,17,18-hexahydrocyclononal [1,2-b:4,5-b′:7,8-b″]triindole (CT), or pharmaceutically acceptable salts, solvates or hydrates thereof, preferably Indole 3 carbinole (I3C) and/or 3,3′ diindolylmethane (DIM), or pharmaceutically acceptable salts, solvates or hydrates thereof.

8. The method of claim 1 wherein
the selective estrogen receptor modulator and/or aromatase inhibitor (A) is Tamoxifen, or a pharmaceutically acceptable salt, solvate or hydrate thereof; and
the estrogen modulator (B) is Indole-3-carbinole (I3C), or a pharmaceutically acceptable salt, solvate or hydrate thereof.

9. The method of claim 1 wherein:
the selective estrogen receptor modulator and/or the aromatase inhibitor (A), and
the estrogen modulator (B)
are administered simultaneously, separately, or sequentially.

10. The method of claim 1 wherein said selective estrogen receptor modulator and/or aromatase inhibitor is administered at a dosage of 1-100 mg/day.

11. The method of claim 1 wherein said estrogen modulator (B) is administered at a dosage of 1-1000 mg/day.

12. The method of claim 1 wherein said
estrogen receptor modulator and/or aromatase inhibitor (A) and/or
estrogen modulator (B);
are administered orally, parenterally, transdermally, topically or enterally, preferably in capsule of tablet form.

13. A method of preventing or treating gynecomastia and/or mastalgia in a subject in need thereof,
conprising administering (A) a selective estrogen receptor modulator and/or aromatase inhibitor in combination with
(B) a cruciferous vegetable, wild nettle root, chrysin, soy, turmeric, maca, flavonoids or grape seed supplement or extract.

14. A composition comprising
(A) a selective estrogen receptor modulator and/or an aromatase inhibitor;
(B) an estrogen modulator, wherein said estrogen modulator is different to said selective estrogen receptor modulator;
for use in the treatment or prevention of gynecomastia and/or mastalgia.

15. A pharmaceutical composition for combination therapy for treating gynecomastia and/or mastalgia, comprising
(A) a first pharmaceutical component comprising a selective estrogen receptor modulator and/or an aromatase inhibitor;
(B) a second pharmaceutical component comprising an estrogen modulator, wherein said estrogen modulator is different to said selective estrogen receptor modulator as active ingredients.

16-18. (canceled)

19. A kit comprising
(A) a selective estrogen receptor modulator and/or an aromatase inhibitor; and
(B) an estrogen modulator different to said selective estrogen receptor modulator;
for simultaneous, separate or sequential use in the treatment of gynecomastia and/or mastalgia.

20-21. (canceled)

22. A pharmaceutical composition comprising:
(A) a selective estrogen receptor modulator and/or an aromatase inhibitor, wherein said selective estrogen receptor modulator and/or aromatase inhibitor is a selective estrogen receptor modulator selected from Tamoxifen, Raloxifene, and Clomifene, or pharmaceutically acceptable salts, solvates or hydrates thereof; and
(B) an estrogen modulator different to said selective estrogen receptor modulator, wherein said estrogen modulator is selected from Indole-3-carbinole (I3C), 3,3′-diindolylmethane (DIM), 5,11-dihydroindolo-[3,2-b]carbazole (ICZ), and/or 5,6,11,12,17,18-hexahydrocyclononal [1,2-b:4,5-b′:7,8-b″]triindole (CT), or pharmaceutically acceptable salts, solvates or hydrates thereof.

23. (canceled)

24. A method of reducing the breast size and/or breast pain of a subject, comprising a step of administering:
(A) a selective estrogen receptor modulator and/or an aromatase inhibitor, wherein said selective estrogen receptor modulator and/or aromatase inhibitor is a selective estrogen receptor modulator selected from Tamoxifen, Raloxifene, and Clomifene, or pharmaceutically acceptable salts, solvates or hydrates thereof; and
(B) an estrogen modulator different to said selective estrogen receptor modulator, wherein said estrogen modulator is selected from Indole-3-carbinole (I3C), 3,3′-diindolylmethane (DIM), 5,11-dihydroindolo-[3,2-b]carbazole (ICZ), and/or and 5,6,11,12,17,18-hexahydrocyclononal [1,2-b:4,5-b′:7,8-b″]triindole (CT), or pharmaceutically acceptable salts, solvates or hydrates thereof;
wherein (A) and (B) are administered simultaneously, separately, or sequentially.

25. The method of claim 2 wherein the supplement is a cruciferous vegetable supplement.

26. The method of claim 6 wherein the aromatase inhibitor is testolactone.
